(19) United States
(12) Patent Application Publication
(10) Pub. No.: US 2018/0021460 A1
(43) Pub. Date: Jan. 25, 2018

(54) PROBES AND METHODS FOR DETECTING AMYLOIDOGENIC PROTEINS

(71) Applicant: Academia Sinica, Taipei (TW)

(72) Inventors: Jen-Tse Joseph HUANG, Taipei City (TW); Ruei-Yu HE, Tainan City (TW)

(73) Assignee: Academia Sinica, Taipei (TW)

(21) Appl. No.: 15/628,765

(22) Filed: Jun. 21, 2017

Related U.S. Application Data
(60) Provisional application No. 62/365,433, filed on Jul. 22, 2016.

(51) Int. Cl. A61K 49/00

(52) U.S. Cl. CPC A61K 49/0056

(57) ABSTRACT

Disclosed herein are novel photocontrollable probes and methods for detecting aggregated forms of amyloidogenic proteins in cells, tissues, or organs of a live subject. Results of such detection may be useful for making prognosis on the subject whether he/she suffers from a neurodegenerative disease and/or diabetes mellitus.

### CROSS-REFERENCE TO RELATED APPLICATIONS

This application claims priority to U.S. Provisional Patent Application Ser. No. 62/365,433, filed Jul. 22, 2016, the content of which is hereby incorporated by reference in its entirety.

### BACKGROUND OF THE INVENTION

### 1. Field of the Invention

The present disclosure in general relates to methods and kits for evaluating the aggregated forms of amyloid polypeptides, more particularly, to photocontrollable probes that rapidly induces in vitro or in vivo amyloid deposit upon photolysis. The present disclosure is also related to methods for detecting the presence or absence of amyloidogenic proteins in cells, tissues, and/or organs of a subject using the photocontrollable probes, as well as methods for making prognosis on whether the subject has a disease and/or disorder associated with amyloid deposit.

### 2. Description of Related Art

Evidence demonstrates that the misfolding and accumulation of soluble protein into insoluble proteinaceous aggregates is a hallmark feature among many neurodegenerative diseases. Such cases include the amyloid-β peptide (Aβ) in Alzheimer's disease (AD), α-synuclein in Parkinson's disease (PD), mutant huntingtin in Huntington's disease (HD), and TAR-DNA binding protein (TDP-43) in amyotrophic lateral sclerosis (ALS).

To monitor the amyloid formation process and characterize the amyloid-induced cytotoxicity, aggregation-prone proteins/peptides are frequently used both in vitro and in vivo. However, these proteins/peptides spontaneously polymerize, which hinders the examination of underlying molecular mechanisms. To circumvent this dilemma, different chemical and synthetic strategies have been developed to enhance their solubility and to temporally inhibit aggregation. In those cases, the amyloidogenesis was initiated by exploiting triggers, such as enzymatic digestion, pH changes, redox potential perturbation, and photo-irradiation. However, due to the difficulty of controlling enzymatic cleavage, the physiological pH, and/or the redox potential in the biological context, none of the known applications allows monitoring of amyloidogenesis in live cells.

Thus, there exist in the related art a need of probes and methods for probing amyloidogenesis in live cells.

### SUMMARY OF THE INVENTION

The present disclosure provides a photocontrollabe probe that rapidly induces in vitro or in vivo amyloid deposit upon photolysis. Accordingly, the photocontrollabe probe is useful for evaluating the aggregated forms of amyloid polypeptides, detecting the presence or absence of amyloidogenic proteins in cells, tissues, and/or organs of a subject, and/or making a prognosis on whether the subject suffers from diseases and/or disorders associated with the aggregated amyloidogenic proteins (e.g., amyloid deposit).

Accordingly, the first aspect of the present disclosure is directed to a photocontrollable probe, which comprises in its structure, at least, a cell penetrating moiety, an amyloidogenic moiety, and a photocleavable moiety disposed between the cell penetrating and amyloidogenic moieties and connected thereto; wherein, the photocleavable moiety is labile upon exposure to a radiation having a wavelength between 250 to 1,000 nm.

According to certain embodiments, the cell penetrating moiety comprises at least 2 charged amino acid residues selected from the group consisting of arginine, lysine, histidine, aspartate, glutamate, and a combination thereof.

In one preferred embodiment, the cell penetrating moiety is composed of 8 arginine residues.

According to other embodiments, the cell penetrating moiety is a cell penetrating peptide (CPP) selected from the group consisting of, Tat peptide, penetratin, pVEC, transportan, MPG, Rep-1, MAP, and R₆W₃.

According to certain embodiments, the amyloidogenic moiety is an amyloidogenic peptide derived from amyloid-β (Aβ), amyloid islet protein, α-synuclein, mutant huntingtin, chromosome 9 open-reading frame 72 (C9orf72) dipeptide repeat protein (DRP), or TAR-DNA binding protein 43 (TDP-43).

In one preferred embodiment, the amyloidogenic peptide is derived from TDP-43 and has a sequence identity at least 90% to SEQ ID NO: 2 (MGGGMNFGAFSINPAM).

In another preferred embodiment, the amyloidogenic peptide is derived from C9orf72 DRP, and may be a poly(glycine-alanine) (poly-GA) fragment, a poly(glycine-proline) (poly-GP) fragment, or a poly(glycine-arginine) (poly-GR) fragment.

In a further embodiment, the amyloidogenic peptide is derived from mutant huntingtin, and may be a polyglutamine (PolyQ) fragment.

In optional embodiments, the amyloidogenic peptide is further conjugated with polyethylenimine (PEI).

According to certain embodiments, the photocleavable moiety comprises a functional group selected from the group consisting of, arylcabonyl, nitroaryl, coumarin-4-yl-methyl, arylmethyl, a metal-containing group, arylsulfonyl, and a silicon based group.

In some embodiments, the metal-containing group is [M(bpy)ₙ]²⁺, where M is Fe²⁺, Ru²⁺, or Co²⁺; n is 2 or 3; and bpy is 2,2′-bipyridine.

In other embodiments, the photocleavable moiety has the structure of rose Bengal, riboflavin, or cobalamin.

In one preferred embodiment, the photocleavable moiety comprises a nitroaryl group that is cleaved upon exposure to the radiation of 365 nm.

According to optionally embodiments, the photocontrollable probe may further comprise a fluorophore linking moiety disposed at the end of the amyloidogenic moiety for linking a fluorophore thereto. In certain embodiments, the fluorophore linking moiety is a cysteine residue.

Examples for suitable fluorophore that may be linked to the photocontrollable probe include, but are not limited to, aminocoumarin, allophycocyanin (APC), Alexa 488, Alexa 568, CY3, CY5, CY7, APC-CY7 conjugate, fluorescein; FluorX, hydroxycoumarin, lucifer yellow, methoxycoumarin, [2-(4-nitro-2,1,3-benzoxadiazol-7-yl)aminoethyl]trimethylammonium (NBD-TMA), phycoerythrin (PE), PE-CY5 conjugate, PE-CY7 conjugate, rhodamine, tetramethylrhodamine isothiocyanate (TRITC)-amines, and Texas Red amines.

The second aspect of the present disclosure provides a kit for detecting the presence or absence of an amyloidogenic protein in cells, tissues, and/or organs of a subject. The kit comprises a first container containing the present photocontrollable probe that comprises in its structure a fluorophore linking moiety; and a second container containing a fluorophore; wherein upon mixing the photocontrollable probe in the first container and the fluorophore in the second container, the fluorophore is linked to the fluorophore linking moiety of the photocontrollable probe.

The third aspect of the present disclosure provides a method for detecting the presence or absence of an amyloidogenic protein in a tissue or an organ of a subject. The method includes steps of,

(a) treating the tissue or the organ of the subject with the kit of the present disclosure;

(b) irradiating the kit-treated tissue or organ of the step (a) with a light having a wavelength between 250 to 1,000 nm; and

(c) subjecting the irradiated tissue or organ of the step (b) to an imaging analysis;

wherein, if amyloid fibrils were observed in the irradiated tissue or organ during the imaging analysis in the step (c), then the amyloidogenic protein is present in the tissue or the organ of the subject.

According to certain embodiments, in the step (b), the tissue, the organ, or the animal is irradiated with the light having a wavelength of 365 nm.

According to certain embodiments, the amyloidogenic protein is any of amyloid-β (Aβ), amyloid islet protein, α-synuclein, mutant huntingtin, chromosome 9 open-reading frame 72 (C9orf72) dipeptide repeat protein (DRP), or TAR-DNA binding protein 43 (TDP-43).

The fourth aspect of the present disclosure provides a method of making a prognosis based on a biological sample of a subject on whether the subject has a neurodegenerative disease or diabetes mellitus. The method comprises steps of:

(a) subjecting the biological sample of the subject to the treatment of the kit of the present disclosure;

(b) exposing the kit-treated biological sample of the step (a) at a radiation having a wavelength between 250 to 1,000 nm; and

(c) subjecting the irradiated biological sample of the step (b) to an imaging analysis;

wherein, if amyloid fibrils was observed in the irradiated biological sample during the imaging analysis in the step (c), then the subject has the neurodegenerative disease or diabetes mellitus.

According to certain embodiments, the biological sample may be cerebrospinal fluid, blood, plasma or oral mucosa. In one preferred embodiment, the biological sample is oral mucosa.

According to certain embodiments, in the step (b), the biological sample is irradiated with the light having a wavelength of 365 nm.

According to certain embodiments, the neurodegenerative disease is scrapie, amyotrophic lateral sclerosis (ALS), Parkinson disease (PD), Alzheimer disease (AD), Lewy body disease, Huntington disease (HD), Machado-Joseph disease, dentatorubral pallidoluysian atrophy (DRPLA), spinal and bulbar muscular atrophy, spinocerebellar ataxia, or fragile X-associated tremor/ataxia syndrome.

According to certain embodiments, the diabetes mellitus is type II diabetes mellitus.

The details of one or more embodiments of this disclosure are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed.

### BRIEF DESCRIPTION OF THE DRAWINGS

The patent or application file contains at least one drawing executed in colors. Copies of this patent or patent application publication with color drawing(s) will be provided by the Office upon request and payment of the necessary fee.

The accompanying drawings, which are incorporated in and constitute a part of the specification, illustrate various example systems, methods and other exemplified embodiments of various aspects of the invention. The present description will be better understood from the following detailed description read in light of the accompanying drawings, where,

FIG. 1: The design and biophysical properties of photocontrollable probes. (a) The composition of the chemically engineered peptide probes, where R represents the tert-butyl group and ALEXA568 in JHR1 and JHR2, respectively. Upon UV light irradiation, rapid amyloidogenesis would occur as the octaarginine sequence is dissociated from the amyloidogenic sequence TDP-43₃₀₇₋₃₂₂. (b) JHR1 (5 μM) existed as spherical vesicles (indicated by white arrow) before photoactivation in transmission electron microscopy (TEM). (c) Cryo-electron microscopy (Cryo-EM) revealed the bilayer vesicle of JHR1 before photoactivation. (d) Average vesicle size of JHR1 before photoactivation. Size was measured by TEM and dynamic laser light scattering (DLS). Scale bars in (b) and (c), 100 nm.

FIG. 2: The chemical properties of photocontrollable probes. (a) A 24 hr time-course TEM analysis of JHR1 (50 μM) with photoactivation is shown. The representative images from the 0, 4^{th} 24^{th} hrs are presented, respectively. Scale bars, 200 nm. The secondary structures of JHR1 (50 μM) revealed by circular dichroism in the absence (b) and presence (c) of photoactivation are compared. The data at 0^{th}, 2^{nd}, 4^{th}, 6^{th}, 9^{th}, and 24^{th} hr were collected, but only representative data collected at 0^{th}, 2^{nd}, and 24^{th} hr are shown. (d) The turbidity of JHR1 (50 μM) determined by UV-vis absorbance before (▪) and after () photoactivation in a 48 hr time course. The data are collected at 0^{th}, 2^{nd}, 4^{th}, 6^{th}, 9^{th}, 24^{th}, and 48^{th} hrs, respectively. (s.d., n=3). (e) The fluorescence signal (a.u.) of thioflavin T (ThT) in the presence of JHR1 (200 μM) before (solid bars) and after (hatched bars) photoactivation.

FIG. 3: Real-time visualization of in vitro photocontrollable nanofibril formation. (a) JHR1 was UV illuminated (335-379 nm, 8.24 mW/cm², 3 min) and recorded in real-time by time-lapse TIRF microscopy with ThT staining for 5 hr (at 5 min intervals). The representative images are shown in 50 min intervals. JHR1 (3 μM) is prepared in the solution containing 10 mM HCl and 10% HFIP (v/v) with ThT (100 μM). Among all of the images, the four representative nanofibril formation patterns can be summarized as (b) unidirectional elongation and growth, (c) multidirectional branching of the budding termini, (d) linkage and fusion of the two growing ends, and (e) meeting of the detour fibrillar ends. The newly growing nuclei, ends, and conjunctions are highlighted with white arrowheads. (f) The superimposed images from the 120^{th} min (green) and 300^{th} min (red) reveal the newly elongating nanofibril network (red) from the existing nanofibrils (yellow, merged) over the past 5 hr. Scale bars, 1 μm. The distribution of the (g) fibrillar length and (h) growth rate are summarized over time. The nanofibril length and growth rate were determined by applying NIS-Elements AR (Nikon) software.

FIG. 4: The photocontrollable probe “seeds” are colocalized with cytosolic EGFP-TDP-43 aggregates. (a) Time course TEM analysis of JHR2 with photolysis is compared. The representative images from the 0, 4^{th}, 24^{th} hrs are shown, respectively. Scale bars, 200 nm. The cytosolic distribution of EGFP-TDP-43 (green) in the JHR2 (red)-treated cells in the absence (b) and presence (c) of photolysis are shown and compared. N2A cells harboring EGFP-TDP-43 are pretreated with AICAR (1 mM, overnight) and then treated with JHR2 (1 μM, 3 hr) before UV illumination and further incubation for 24 hr. Cell nuclei are stained with Hoechst (blue), and the cell morphology is shown in the DIC channel. The aggregates (arrowhead) from EGFP-TDP-43 (green), JHR2 (red), or both (yellow) in (c) were magnified in insets. Scale bars, 10 μm.

FIG. 5: Photocontrollable probe allows spatiotemporal visualization of amyloidogenesis, induces TDP-43 aggregation. (a) The distribution of the endogenous TDP-43 (green) in the JHR1-treated (upper two panels) or control (lower two panels) cells with pretreated AICAR (1 mM, 24 hr) in the absence and presence of photoactivation. After JHR1 treatment and UV illumination, the cells are incubated for 24 hr, fixed and immunostained with anti-TDP-43 antibody. Inset: The blown up image shows the TDP-43 aggregates (arrowheads) observed in the JHR1-treated cell upon illumination (+JHR1+Light). In the untreated (−JHR1−Light), UV-irradiated only (−JHR1+Light), and JHR1-treated only (+JHR1−Light) cells, little or no endogenous TDP-43 aggregate was observed. Cell nuclei are stained with Hoechst (blue). (b) The cytoplasmic puncta observed in (a) are quantitated with high content screening analysis (Yokogawa). ** represents p<0.01. (c) The patterned (hence cell-selective) photoactivation, masked from a pinhole in the light path, is illustrated by using Hoechst stained cells. When the pinhole was not applied (upper panel: illuminated without a light mask), all cells were fluorescent. When a pinhole was applied to confine the illuminated area, only the cells within this region (circumscribed by a white dashed line) were irradiated and emitted fluorescence (lower panel). (d) The JHR2-treated cells outside (left side to the dashed line) and inside (right side to the dashed line) the illuminated area are shown and compared. Inset: The blown up image shows the aggregates (arrowhead) from endogenous TDP-43 (green), JHR2 (red), or both (yellow). Bars, 10 μm. (e) Time-lapse monitoring of the EGFP-TDP-43 (green) ectopically expressed in N2A cells in the presence of JHR2 after photolysis. Inset: The blown up images of the EGFP-TDP-43 (green) aggregates at the 20^{th} hr and 24^{th} hr after photolysis (arrowheads). (f) The endpoint (t=25^{th} hr) of (e). While both the EGFP-TDP-43 (green) and JHR2 (red) show aggregates in (f), Annexin V staining reveals that these cells are dying (orange). Cell nuclei are stained with Hoechst (blue), and the cell morphology is shown in the DIC channel. The concentration of JHR2 and the photolysis conditions described in (a) are also used in (b-f). Scale bars, 10 μm.

FIG. 6: Mislocalization of endogenous Ran, TDP-43, and importin-α1 initiated by JHR2 aggregation. (a) The distribution of the endogenous Ran (green) in the JHR2 (orange)-treated (upper two panels) or control (lower two panels) cells in the absence and presence of photoactivation. It should be noted that Ran redistributes to the cytosol with the formation of cytosolic JHR2 aggregates upon illumination (+JHR2+Light). In the untreated (−JHR2−Light), UV-irradiated only (−JHR2+Light), and JHR2-treated only (+JHR2−Light) cells, no endogenous Ran redistribution was observed. (b) The distribution of the endogenous TDP-43 (green) and importin-α1 (purple) in the JHR2 (orange)-treated (upper two panels) or control (lower two panels) cells in the absence and presence of photoactivation. TDP-43 and importin-α1 mainly remains in the nucleus in the untreated (−JHR2−Light), UV-irradiated only (−JHR2+Light), and JHR2-treated only (+JHR2−Light) cells. TDP-43 and importin-α1 translocate from nucleus to cytosol in the presence of JHR2 and photoactivation (+JHR2+Light), and colocalized with the JHR2 aggregates (indicated by arrowheads). Scale bars, 10 μm.

### DETAILED DESCRIPTION OF THE INVENTION

The detailed description provided below in connection with the appended drawings is intended as a description of the present disclosure and is not intended to represent the only forms in which the present disclosure may be constructed or utilized.

### 1. Definitions

Unless otherwise indicated, the term “polypeptide” refers to a polymer of amino acid residues joined by peptide bonds, whether produced naturally or in vitro by synthetic means. Polypeptides of less than around 50 amino acid residues in length are herein referred to as “peptides.” The term “polypeptide” as used herein denotes the product of a naturally occurring polypeptide, precursor form or pro-protein. Polypeptides can also undergo maturation or post-translational modification processes that may include, but are not limited to: glycosylation, proteolytic cleavage, lipidization, signal peptide cleavage, pro-peptide cleavage, phosphorylation, and such like. The term “protein” is used herein to refer to a large polypeptide molecule as well as a macromolecule comprising one or more polypeptide chains.

As discussed herein, minor variations in the amino acid sequences of polyepeptides/peptides are contemplated as being encompassed by the presently disclosed and claimed inventive concept(s), providing that the variations in the amino acid sequence maintain at least 90% sequence identity, such as at least 70%, 71%, 72%, 73%, 75%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% sequence identity. Peptides or polypeptides of the present disclosure may be modified specifically to alter a feature of the peptide unrelated to its physiological activity. For example, certain amino acids can be changed and/or deleted without affecting the physiological activity of the peptide in this study (i.e., its ability to treat inflammation related diseases and/or conditions). In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the peptide derivative. Fragments or analogs of proteins/peptides can be readily prepared by those of ordinary skill in the art. Preferred amino- and carboxy-termini of fragments or analogs occur near boundaries of functional domains. In one example, one amino acid residue (e.g., valine) of the present synthetic peptide is conservatively replaced (e.g., by leucine). In other examples, two amino acid residues of the present synthetic peptide are conservatively replaced by other suitable amino acid residues, for example, valine (V) and arginine (R) are replaced by the pair of amino acids that includes, but is not limited to, methionine (M) and lysine (K), lysine (K) and proline (P), tryptophan (W) and isoleucine (I), isoleucine (I) and proline (P), asparagine (N) and valine (V), and glutamine (G) and lysine (K).

“Percentage (%) sequence identity” is defined as the percentage of amino acid residues in a candidate sequence that are identical with the amino acid residues in the specific peptide sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two amino acid sequences was carried out by computer program Blastp (protein-protein BLAST) provided online by Nation Center for Biotechnology Information (NCBI). The percentage amino acid sequence identity of a given amino acid sequence A to a given amino acid sequence B (which can alternatively be phrased as a given amino acid sequence A that has a certain % amino acid sequence identity to a given amino acid sequence B) is calculated by the formula as follows:

$$\frac{X}{Y} \times 100 \%$$

where X is the number of amino acid residues scored as identical matches by the sequence alignment program BLAST in that program's alignment of A and B, and where Y is the total number of amino acid residues in A or B, whichever is shorter.

Throughout the present disclosure, the positions of any specified amino acid residues within a peptide are numbered starting from the N terminus of the peptide. When amino acids are not designated as either D- or L-amino acids, the amino acid is either L-amino acid or could be either D- or L-amino acid, unless the context requires a particular isomer.

Unless otherwise indicated, the term “amyloidogenic peptide” as used herein refers to a peptide about 10-100 amino acids in length, and is capable of inducing the folding of specific proteins, leading to protein aggregation and the appearance of amyloid deposits, which is the key step in the development of various diseases, e.g., neurodegenerative disease and diabetes.

The term “amyloid deposit,” “amyloid fibril,” or “amyloid” may be used interchangeably, and refer generally to insoluble proteinaceous substances with particular physical characteristics independent of composition of proteins or other molecules that are found in the substance. Amyloid fibril may be identified by its amorphous structure, eosinophilic staining, or changes in thioflavin fluorescence. Protein or peptide components of amyloid fibril are termed “amyloidogenic proteins” or “amyloidogenic polypeptides,” and include, but are not limited to, amyloid-β (Aβ), amyloid islet protein, α-synuclein, mutant huntingtin, chromosome 9 open-reading frame 72 (C9orf72) dipeptide repeat protein (DRP), TAR-DNA binding protein 43 (TDP-43), and the like.

Unless otherwise indicated, the term “patient” and “subject” are used interchangeably in the present disclosure, and may be an animal or a human subject.

As used herein, the term “tissue” means an aggregate of cells of a particular kind that form a structural material of an animal, such as skin or mucosal. The term “organ” as used herein refers to a collection of tissues, some of which may be different from one another, which work together to perform a more complex function as if the tissues were a single structure unit. Thus, the term “organ” in the present disclosure means vascularized organ such as brain, heart, liver, kidney and etc. An exemplary and preferred organ is the brain.

Unless otherwise indicated, the terms “treat,” “treating” and “treatment” contemplate an action that occurs while a patient is suffering from the specified disease or disorder, which reduces the severity of the disease or disorder, or one or more of its symptoms, or retards or slows the progression of the disease or disorder.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The singular forms “a”, “and”, and “the” are used herein to include plural referents unless the context clearly dictates otherwise.

### 2. The Photocontrollable Probe of the Present Invention

Aggregation of amyloid polypeptide has been implicated in various amyloid diseases. Although significant advances have resulted in the identification of the misfolded disease proteins, yet the formation of amyloid deposit in live cells remain enigmatic. To help elucidate the process, inventors of this application designed and synthesized a photocontrollable probe, which may prevent or reduce the formation of abnormal protein aggregates or may lead to dissociation of existing abnormal protein aggregates, while penetrates the cell membrane and releases an amyloidogenic peptide therein upon exposure to a radiation of suitable wavelength; preferably, the photocontrollable probe is connected to a fluorophore, thereby allows in situ monitoring of the amyloidogenesis in live cells. Accordingly, the present probe is suitable as a tool for determining the presence or absence of an amyloidogenic protein in cells, tissues, and/or organs of a subject; as well as making a prognosis on whether the subject of the cells, tissues or organs may be suffering from a neurodegenerative disease or diabetes mellitus based on the determination of the tissue sample.

Accordingly, it is the first aspect of the present disclosure to provide a photocontrollable probe, which comprises in its structure, at least, a cell penetrating moiety, an amyloidogenic moiety, and a photocleavable moiety disposed between the cell penetrating and amyloidogenic moieties and connected thereto; wherein, the photocleavable moiety is labile upon exposure to a radiation having a wavelength between 250 to 1,000 nm.

According to embodiments of the present disclosure, the cell penetrating moiety may be a peptide that comprises at least 2 charged amino acid residues, such as 2, 3, 4, 5, 6, 7, 8, 9, 10, 11, 12, 13, 14, 15, 16, 17, 18, 19, 20, 21, 22, 23, 24, 25, 26, 27, 28, 29 and 30 charged amino acid residues. The charged amino acid residues may exist in a continuous stretch of 2 to 16 residues; alternatively, the charged amino acid residues stretch may be dispersed by non-charged amino acid residues. Examples of charged amino acid residues include, but are not limited to, arginine (R), lysine (K), histidine (H), aspartate (D), glutamate (Q), and a combination thereof. In one preferred example, the cell penetrating moiety is composed of 8 arginine residues (SEQ ID NO: 1, RRRRRRRR). In another example, the cell penetrating moiety is a cell penetrating peptide (CPP) well known in the related art. The CPP may be selected from the group consisting of, Tat peptide (SEQ ID NO: 3, GRKKRRQRRRPPQ), penetratin (SEQ ID NO: 4, RQIKIWFQNRRMKWKK), pVEC (SEQ ID NO: 5, LLIILRRRIRKQAHAHSK), transportan (SEQ ID NO: 6, GWTLNSAGYLLGKINLKALAALAKKIL), MPG (SEQ ID NO: 7, GALFLGFLGAAGSTMGAWSQPKKKRKV), Rep-1 (SEQ ID NO: 8, KETWWETWWTEWSQPKKKRKV), MAP (SEQ ID NO: 9, KLALKLALKALKAALKLA) and R₆W₃ (SEQ ID NO: 10, RRWWRRWWRR).

According to embodiments of the present disclosure, the amyloidogenic moiety may be a fragment derived from an amyloidogenic protein, such as amyloid-β (Aβ), amyloid islet protein, α-synuclein, mutant huntingtin, chromosome 9 open-reading frame 72 (C9orf72) dipeptide repeat protein (DRP), or TAR-DNA binding protein 43 (TDP-43). In some embodiments, the amyloidogenic moiety is an amyloidogenic peptide derived from TDP-43, and has an amino acid sequence at least 70% identical to SEQ ID NO: 2 (MGGGMNFGAFSINPAM), such as 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% A or 100% identical to SEQ ID NO: 2; preferably, the amyloidogenic peptide derived from TDP-43 has an amino acid sequence at least 80% identical to SEQ ID NO: 2 (MGGGMNFGAFSINPAM), such as 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 2; more preferably, the amyloidogenic peptide derived from TDP-43 has an amino acid sequence at least 90% identical to SEQ ID NO: 2 (MGGGMNFGAFSINPAM), such as 90%, 91%, 92%, 93%, 94%, 95%, 96%, 97%, 98%, 99% or 100% identical to SEQ ID NO: 2. In one preferred embodiment, the amyloidogenic moiety has the amino acid sequence of SEQ ID NO: 2. In other embodiments, the amyloidogenic moiety is an amyloidogenic peptide derived from C9orf72 DRP, and may be any of a poly(glycine-alanine) (poly-GA) fragment, a poly(glycine-proline) (poly-GP) fragment, or a poly(glycine-arginine) (poly-GR) fragment. In still further embodiments, the amyloidogenic moiety is an amyloidogenic peptide derived from mutant huntingtin. Preferably, the amyloidogenic peptide derived from mutant huntingtin is a polyglutamine (PolyQ) fragment. According to optional embodiments, any of the amyloidogenic peptide described above may be further conjugated with polyethylenimine (PEI).

According to embodiments of the present disclosure, the cell penetrating moiety and the amyloidogenic moiety of the photocontrollable probe are respectively linked to a photocleavable moiety that is labile upon exposure to a radiation having a wavelength between 250 to 1,000 nm, such as 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 900, 905, 910, 915, 920, 925, 930, 935, 940, 945, 950, 955, 960, 965, 970, 975, 980, 985, 990, 995, and 1,000 nm. In some embodiment, the photocleavable moiety is cleaved upon exposure to a radiation between 250 to 400 nm, such as 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400 nm. In other embodiments, the photocleavable moiety is cleaved upon exposure to a radiation between 700 to 1,000 nm, such as 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 900, 905, 910, 915, 920, 925, 930, 935, 940, 945, 950, 955, 960, 965, 970, 975, 980, 985, 990, 995, and 1,000 nm. According to embodiments of the present disclosure, the photocleavable moiety comprises a functional group selected from the group consisting of, arylcabonyl, nitroaryl, coumarin-4-yl-methyl, arylmethyl, a metal-containing group, arylsulfonyl, and a silicon based group (e.g., tris(trimethylsilyl) group). In some embodiments, the metal-containing group is [M(bpy)ₙ]²⁺, where M is Fe²⁺, Ru²⁺, or Co²⁺; n is 2 or 3; and bpy is 2,2′-bipyridine. In other embodiments, the photocleavable moiety has the structure of rose Bengal, riboflavin, or cobalamin. In one preferred embodiment, the photocleavable moiety comprises the nitroaryl group, and is cleaved upon exposure to the radiation of 365 nm.

Optionally, the present photocontrollable probe further includes a fluorophore linking moiety disposed at the end of the amyloidogenic moiety and connected thereto. The fluorophore linking moiety may be an amino acid residue having a reactive side chain suitable for coupling with a fluorophore, so that the amyloidogenic process initiated by the amyloidogenic moiety of the present probe may be followed in situ in live cells. In one preferred example, the fluorophore linking moiety is a cysteine residue, in which the sulfhydryl group (—SH) of the cysteine residue reacts with a fluorophore (e.g., a fluorescent dye) via the sulfhydryl-maleimide reaction, thereby conjugates the fluorophore thereto. Examples of the fluorophore suitable for use in the present invention include, but are not limited to, aminocoumarin, allophycocyanin (APC), Alexa 488, Alexa 568, CY3, CY5, CY7, APC-CY7 conjugate, fluorescein, FluorX, hydroxycoumarin, lucifer yellow, methoxycoumarin, [2-(4-nitro-2,1,3-benzoxadiazol-7-yl)aminoethyl]trimethylammonium (NBD-TMA), phycoerythrin (PE), PE-CY5 conjugate, PE-CY7 conjugate, rhodamine, tetramethylrhodamine isothiocyanate (TRITC)-amines, and Texas Red amines. According to preferred embodiments, Alexa 560 is coupled to a cysteine residue disposed at the end of the amyloidogenic peptide.

The present disclosure also encompasses a kit for detecting the presence or absence of an amyloidogenic protein in a biological sample, such as cerebrospinal fluid, blood, plasma or oral mucosa. The kit comprises a first container, which contains the present photocontrollable probe, particularly the probe having a fluorophore linking moiety; and a second container, which contains a fluorophore; wherein, upon mixing the photocontrollable probe in the first container and the fluorophore in the second container, the fluorophore is linked to the fluorophore linking moiety of the photocontrollable probe, allowing the user to follow the amyloidogenic process in situ. The kit may further comprise a legend associated with the first and second containers, for directing the user how to use the kit to detect the present or absence of the amyloidogenic protein in the biological sample. The legend may be in a form of pamphlet, tape, CD, VCD or DVD. The kit may further comprise a negative control that indicates the normal level of the amyloidogenic protein in a healthy subject.

### 3. Method of Use

The present invention thus encompasses a method for detecting the presence or absence of an amyloidogenic protein in a tissue or an organ of a subject. The method includes steps of,

(a) subjecting the tissue or the organ to the treatment of the kit of the present disclosure;

(b) irradiating the kit-treated tissue or organ of the step (a) with a light having a wavelength greater than 250 nm; and

(c) subjecting the irradiated tissue or organ of the step (b) to an imaging analysis;

wherein, if amyloid fibrils were observed in the irradiated tissue or organ during the imaging analysis in the step (c), then the amyloidogenic protein is present in the tissue or the organ of the subject.

According to certain embodiments, in the step (b), the kit-treated tissue or organ is irradiated with the light having a wavelength of 365 nm.

According to certain embodiments, the amyloidogenic protein is any of amyloid-β (Aβ), amyloid islet protein, α-synuclein, mutant huntingtin, chromosome 9 open-reading frame 72 (C9orf72) dipeptide repeat protein (DRP), or TAR-DNA binding protein 43 (TDP-43).

The present disclosure also provides a method of making a prognosis on whether a subject has a neurodegenerative disease or diabetes mellitus. The prognosis is made to a biological sample of the subject, which includes but is not limited to, cerebrospinal fluid, blood, plasma or oral mucosa. The method comprises steps of:

(a) subjecting the biological sample of the subject to the treatment of the kit of the present disclosure;

(b) exposing the kit-treated biological sample of the step (a) at a radiation having a wavelength between 250 to 1,000 nm; and

(c) subjecting the irradiated biological sample of the step (b) to an imaging analysis;

wherein, if amyloid fibrils were observed in the irradiated biological sample during the imaging analysis in the step (c), then the subject has the neurodegenerative disease or diabetes mellitus.

According to certain embodiments, in the step (b), the biological sample is irradiated with the light having a wavelength of 365 nm.

According to certain embodiments, the neurodegenerative disease is scrapie, amyotrophic lateral sclerosis (ALS), Parkinson disease (PD), Alzheimer disease (AD), Lewy body disease, Huntington disease (HD), Machado-Joseph disease, dentatorubral pallidoluysian atrophy (DRPLA), spinal and bulbar muscular atrophy, spinocerebellar ataxia, or fragile X-associated tremor/ataxia syndrome. In preferred embodiments, the present method is useful for making prognosis to subjects whether they suffer from or suspected of having AD.

According to certain embodiments, the diabetes mellitus is type II diabetes mellitus. In some embodiments, the present method is useful for making prognosis to subjects whether they suffer from or suspected of having type II diabetes mellitus.

The present invention will now be described more specifically with reference to the following embodiments, which are provided for the purpose of demonstration rather than limitation. While they are typically of those that might be used, other procedures, methodologies, or techniques known to those skilled in the art may alternatively be used.

### EXAMPLES

Materials and Methods

Polypeptide Synthesis.

Both amino acids and 4-{4-[1-(9-fluorenylmethyloxycarbonylamino)ethyl]-2-methoxy-5-nitrophenoxy}nb ut-anoic acid (Fmoc-photolabile linker) were purchased from Advanced ChemTech. Peptides were synthesized by the standard FMOC polyamide chemistry on Rink amide AM resin using the peptide synthesizer (PS3, Rainin Instrument, USA). After cleavage from the resin, crude polypeptides were purified by high-performance liquid chromatography (HPLC) (1260 Infinity LC system, Agilent, USA) equipped with a C18 reverse phase semi-preparative column (Shiseido, Japan). The gradient separation was achieved by mixing Buffer A (5% acetonitrile/0.1% TFA/94.9% water) and Buffer B (0.1% TFA/99.9% acetonitrile). The flow rate was kept at 3 mL/min. Polypeptide purity was confirmed by RP-HPLC with an analytical column (C18) and identified by matrix-assisted laser desorption/ionization (MALDI) (Applied Biosystem, USA) mass spectroscopy.

Transmission Electron Microscopy

JHR1 and JHR2 (5 or 50 μM) were prepared in PBS buffer (14 mM KCl, 10 mM sodium phosphate, pH=7.5) irradiated with or without UV, respectively (365 nm UV LED, 32 mW/cm², 60 secs) and then incubated at 37° C. for 24 hr. The resulting aliquots of peptide solutions (5 μL) were applied on glow-charged 300 mesh formvar- and carbon-coated copper grids stained with 1% uranyl acetate. After drying overnight, all samples were analyzed using a JEM-2011 electron microscope (JEOL, Japan).

Dynamic Light Scattering

The particle size was calculated from the Brownian motion of the particles using the Stokes-Einstein equation. The method yields a hydrodynamic diameter, which is a calculated particle diameter of a sphere that has the same measured motion in the solute as the actual particle. JHR1 (5 μM) was prepared in H₂O without UV. The size of JHR1 vesicle was measured using dynamic light scattering (DLS) with a zetasizer (Malvern Zetasizer Nano ZS, Malvern Instruments, Worcestershire, UK). The analyses was performed with He—Ne Laser (633 nm) at scattering angle of 175° at 25° C. Size measurements were done in triplicate for each sample.

Cryo-Electron Microscopy

For cryo-EM, JHR1 (5 μM) was prepared in H₂O without UV. 4 μL of each prepared sample was deposited onto a glowdischarged 200-mesh Quantifoil holy carbon grid (1.2×1.3 μm hole size, Quantifoil Micro Tools). The grid was flash frozen in liquid ethane using a Vitrobot (FEI).

Circular Dichroism Measurements

The peptide solutions (50 μM) were prepared in PBS as described above, and UV-irradiation was carried out by a 365 nm UV LED lighting spot curing for hard drives (UVATA, China, 32 mW/cm², 60 secs). The CD spectra of the resulting peptide solutions were measured with a 1 mm quartz cuvette on the J-815 CD spectrometer (JASCO, Japan). All data were collected from 195 to 260 nm with a scanning speed of 100 nm/min. Three scans were averaged for each sample.

UV-Vis Spectrophotometry

The peptide solution (50 μM) was prepared in PBS as described above and recorded with a 1 cm quartz cuvette on the DU800 spectrophotometer (Beckman, America). All turbidity data were collected at 600 nm.

Thioflavin T (ThT) Binding Assay

The ThT working solution (200 μM ThT in PBS) was freshly prepared and filtered through a 0.22 μm Millipore filter. A 40 μL aliquot of the peptide solution (200 μM JHR1 in PBS buffer) with or without UV-irradiation (365 nm UV LED, UVATA, China, 32 mW/cm², 60 secs) was mixed with 40 μL of the ThT working solution for 5 min at room temperature. The fluorescence emission spectra at 480 nm was taken in a 3 mm path length rectangular fluorescence quartz cuvette on a F-4500 spectrofluorimeter (HITACHI, Japan) with excitation at 442 nm.

Synthesis of the Alexa Fluor 568-Labeled Peptide (JHR2)

Two microliters of tris(2-carboxyethyl)phosphine (TCEP) solution (0.5 M) in PBS buffer was added into JHR1 (100 μM in PBS buffer) for 30 minutes to remove the Cys side-chain (Stbu) protecting group at room temperature. Then, 1 equiv of Alexa Fluor 568® label in DMSO was added for 90 min for fluorophore labeling.

Time-Lapse TIFR Imaging for In Vitro Amyloidogenesis

Time-lapse TIRF and epifluorescence image collection were carried out using a Nikon TiE microscope, where samples were illuminated with an ultra-high pressure 130 W mercury lamp for the photolysis reaction and a 405 nm laser light source for the ThT excitation. For ThT staining, JHR1 (6 μM) was prepared in 10 mM HCl and 10% hexafluoroisopropanol (HFIP) (v/v) mixed with the same amount of ThT (200 μM in PBS buffer) solution. An aliquot of 200 μL was mounted on a 35-mm glass bottom dish (Ibidi, Germany) and was cap sealed to prevent the loss of the liquid. After photolysis (335-379 nm, 8.24 mW/cm², 5 min by mercury lamp), images were acquired for 6 hr at 5-min intervals by time-lapse TIRF microscopy with a 405 nm laser. The ThT signals were filtered with an ECFP cube (Chroma) and collected by an Andor iXon3 888 back-illuminated high sensitivity EMCCD camera. The fibril length was determined by Nikon NIS element software, and the growth rate was calculated by dividing the length by the corresponding growth time.

Cell Maintenance, Peptide Treatment, and Photolysis

Mouse neuroblastoma N2A cells (generous gift from Dr. Yijuang Chern, IBMS, Academia Sinica) were cultured in Dulbecco's modified Eagle's medium (DMEM) (Invitrogen) supplemented with 2 mM glutamine, 10% heat-inactivated fetal bovine serum and 100 U/mL penicillin-streptomycin (Invitrogen) at 37° C. in a humidified atmosphere with 5% CO₂. Before peptide incubation, cells were treated with 5-aminoimidazole-4-carboxamide-1-β-D-ribofuranoside (AICAR) to induce the cytoplasmic mislocalization of TDP-43. JHR2 was prepared by dissolving the lyophilized powder in the cell culture medium. Cells were incubated with polypeptide-containing medium for 3 hr, washed, and UV-illuminated (mercury lamp with 345-385 nm bandpass filter, average power: 8.24 mW/cm²).

Confocal and Super-Resolution Fluorescence Microscopy with Airy Scan

For EGFP-TDP-43 expression, 2×10⁵ of N2A cells in a 30-mm square coverslip were transfected with 1 μg of full-length TDP-43-EGFP plasmid with TurboFect Transfection Reagent (Thermo Scientific) according to the manufacturer's protocol. After peptide and photolysis treatment as described above, cells were incubated for an additional 24 hr and then fixed with 4 paraformaldehyde. Confocal images were captured with LSM 880 and super resolution imaging was performed with Airy Scan (Zeiss, Germany). For immunostaining, anti-TDP-43 antibody (Abcam, ab104223) and anti-Ran (Abcam, ab155103) antibody were used, respectively.

Time-lapse Living Fluorescence Cell Microscopy and Annexin V Staining

N2A cells (5×10⁴) were transfected with 1 μg of full-length TDP-43-EGFP in a sterilized 35 mm μ-Dish (Ibidi, Martinsried, Germany). The living cells were imaged with a Nikon TiE inverted microscope equipped with a Tokai-hit constant humidity chamber (37° C., 5% CO₂) for 25 hr. Hoechst, EGFP, Aleax Fluor 568 and Cy5 cube filters were used to collect the fluorescence emission accordingly. Cellular images were captured with an Andor iXon3 888 back-illuminated high sensitivity EMCCD camera. Images were analyzed and quantified using Nikon NIS element software. After 24 hr of peptide treatment, the cells were washed twice with PBS, washed once with 1× Annexin V binding buffer, incubated with Annexin V-Cy5 (BioVision, USA) at room temperature and imaged.

Microinjection

The glass microneedles for microinjection (1B100-F4, World Precision Instruments, USA) were pulled with a PC-10 capillary puller (Narishige, Japan). Before microinjection, microneedles were back-loaded with JHR1 peptide solution (10 μM) in PBS. A Picospriterzer III microinjection system and a joystick micromanipulator (Narishige, Japan) mounted with a stage-side of a microscope (IX-71, Olympus, Japan) were used for delivering the peptide solution into the cytosol of N2A cells. After microinjection (injection pressure, 80 hPa; injection time, 100 msec; compensation pressure, 20 hPa), cells were subsequently rinsed with 2 mL of L-15 medium supplemented with 10% FBS for 1 hr of recovery.

Spatial Activation of the Polypeptides in Living Cells

For the spatial resolution studies, the UV (335-379 nm) light spot in the view field was confined to a diameter of approximately 50 μm with a pinhole (1 mm in diameter) positioned after the light guide of the metal halide lamp. After the spatially confined UV photolysis, the cells were incubated at 37° C. for 24 hr in the culture medium as described above before fixation.

JHR2 Treatments in Mouse Cortical Neurons

Primary culture of mouse cortical neurons was carried out as previously described (Chen et al., Neurosci Res 70, 118-123 (2011)). The JHR2 peptide (final concentration, 1 μM) was dissolved in the pre-equilibrated neuronal maintenance medium containing half of the old cultured medium for treatments. The 21 days in vitro (21 DIV) cortical neurons were incubated with null or JHR2-containing medium for 8 hr. To completely remove the free JHR2 in the medium, the washing process, involving replacement with new 500 μL pre-equilibrated neuronal maintenance medium, would be applied twice. Cortical neurons were fixed 24 hr after UV-irradiation (380 nm UV, average power 18 mW/cm², 2 min mercury lamp). Hippocampal neurons were fixed with 3.7 formaldehyde in 1×PBS at 37° C. for 15 min, followed by membrane permeabilization with 0.25% Triton X-100 for 5 min at room temperature. Then, they were blocked with 10% BSA for 30 min at 37° C. Neurons were incubated for 1 hr at 37° C. with primary antibodies (anti-TDP-43, 1:1000, Abcam, ab104223; anti-β-III-tubulin, 1:200, Abcam, ab18207) in 2% BSA. Alexa Fluor 488-labeled (anti-mouse) and 680-labeled (anti-rabbit) secondary antibodies (1:1000, Life Technologies) were incubated for 1 hr at 37° C. in the dark. Fluorescence images were acquired with a Nikon Eclipse-Ti inverted microscope equipped with a Photometrics CoolSNAP HQ2 CCD camera.

Statistical Analysis

Statistical analysis was performed using one-way analysis of variance by Student's t-test. Significance was accepted at p<0.05.

### Example 1 Synthesis of the Photocontrollable Probe

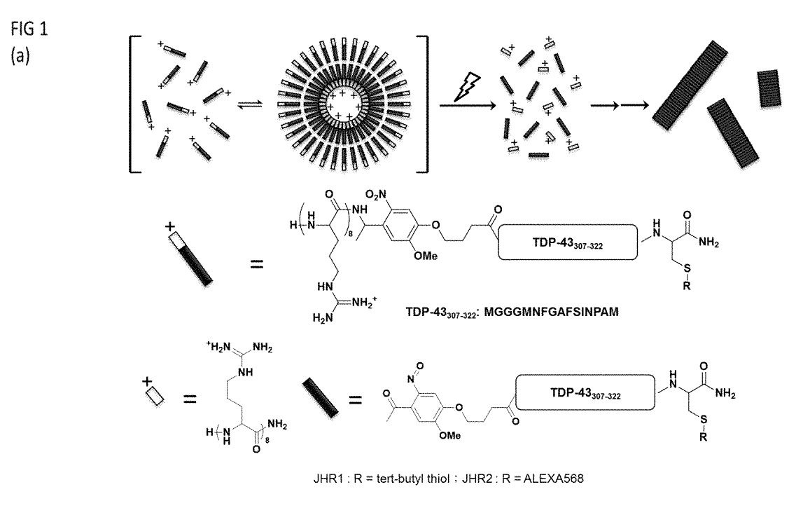

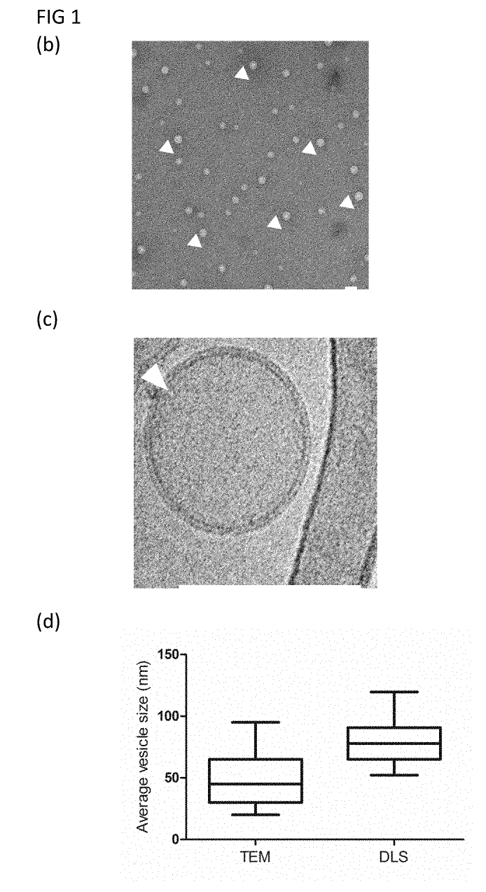

Two probes JHR1 and JHR2 were synthesized and purified in accordance with the procedures described in the “Materials and Methods” section. Specifically, each JHR1 and JHR2 comprised in its structure, an eight-arginine (8R) sequence (SEQ ID NO: 1, RRRRRRRR), and the amyloidogenic sequence derived from TDP-43 (SEQ ID NO: 2, MGGGMNFGAFSINPAM, which was the amino acid residues 307 to 322 of TDP-43), and an o-nitrobenzyl photocleavable moiety disposed between the 8R peptide (SEQ ID NO: 1) and the amyloidogenic sequence (SEQ ID NO: 2). Further, JHR1 and JHR2 respectively comprised a tert-butyl thiol and a fluorophore (i.e., Alexa568), linked to the cysteine residue of the amyloidogenic sequence of TDP-43 (FIG. 1, panel a). Both JHR1 and JHR2 were purified by the reverse phase HPLC and characterized by ESI and MALDI mass spectroscopy (data not shown).

### Example 2 Characterization of the Photocontrollable Probe of Example 1

2.1 In Vitro Size Characterization of JHR1

It was found that JHR1 probe (5 μM) could self-assemble into spherical vesicles (FIG. 1, panel b, indicated by the arrows) before being photolysis by transmission electron microscopy (TEM) with the average vesicle size around 48±20 nm. It was hypothesized that the vesicle structure is stabilized by the amphiphilic properties in JHR1 (e.g., Columbic repulsion and hydrophobic/π-π interactions) (FIG. 1, panel a). The bilayer structure of the vesicle was confirmed by Cryo-electron microscopy (Cryo-EM) (FIG. 1, panel c). The mean hydrodynamic diameter (average vesicle size) of JHR1 as characterized by dynamic light scattering (DLS) was 80±21 nm, with a low polydispersity index (>0.3), indicating the formation of monodispersed nano-vesicles (FIG. 1, panel d). The vesicle size of JHR1 is better reflected by hydrodynamic diameter using DLS rather than TEM, which is conducted under dehydrated, vacuum environment.

2.2 Photo Irradiation Promoted Nanofibril Formation with Strong Amyloid Properties

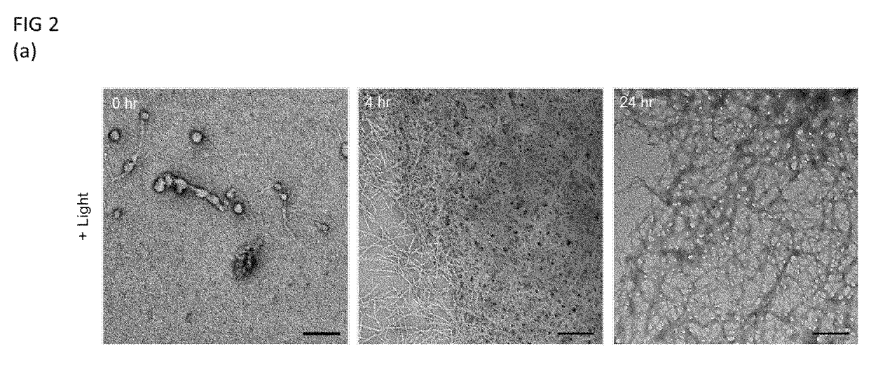

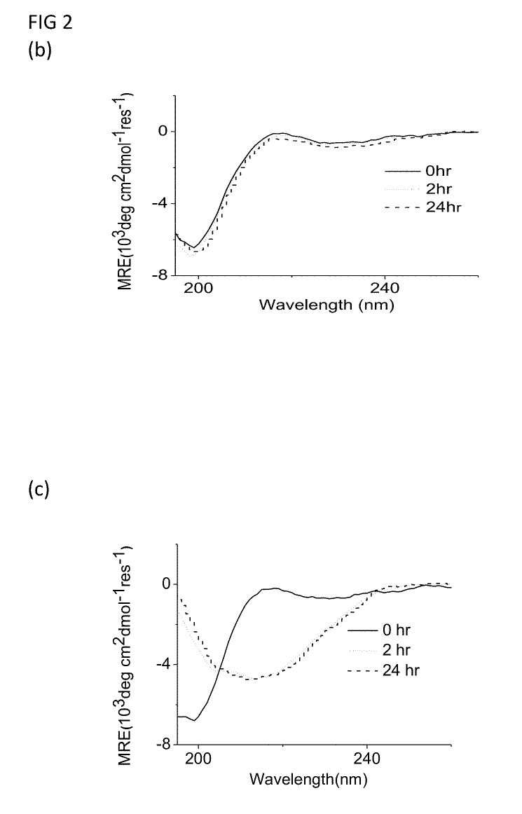

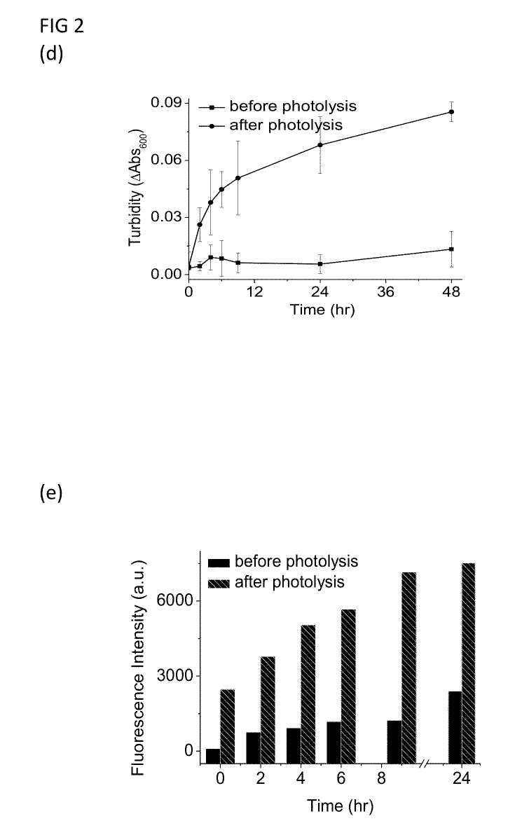

A 24 hr time-course TEM images of JHR1 probe (50 μM) nanofibril formation was observed upon irridation (wavelength: 365 nm, power density: 32 mW/cm², duration: 60 secs) (FIG. 2, panel a). In addition to fibrillization, a β-sheet-rich secondary structure is another important feature of amyloid, which can be characterized by circular dichroism (CD) spectroscopy. As expected, the conformation of JHR1 remained mostly random coil in the dark, the measurements were taken over a period of 24 hr, and only data collected at 0, 2, and 24 hr were illustrated in FIG. 2, panel b. However, upon exposure to light in PBS for 2 hr, the molar ellipticity at 200 nm increased drastically along with a reduction at 218 nm (FIG. 2, panel c), which indicated a structural transformation of JHR1 from mostly random coils to β-sheets. Since no further changes in either the CD spectra or TEM after photoactivation, which indicated that the β-sheet-rich nanofibrils formation was speeded up by irradiation and promptly reached equilibrium. Similarly, JHR1 remained mostly soluble in the dark but exhibited a time-dependent increase in the visible turbidity 48 hr after photoactivation (FIG. 2, panel d). These results suggest that JHR1 has an extremely short nucleation/lag phase and a moderate elongation phase (approximately 10 hr) after photoactivation. Finally, thioflavin T (ThT), a probe that may be intercalated into the amyloid cross-β structure and thereby gives rise to enhanced fluorescence (Liu et al., Chemical Communications 49, 11212-11214 (2013)) was used to confirm the formation of amyloid. It has been confirmed that ThT would preferentially bind to TDP-43₃₀₇₋₃₂₂ aggregates and enhanced fluorescent emission. In this example, we observed that UV-irradiated JHR1 emitted significant aggregation-induced fluorescence in the presence of ThT after 2 hr (FIG. 2, panel e).

Taken together, data in this example demonstrate that “light” can remotely and efficiently control and promote the “amyloidogenesis process” of the present chemically engineered peptide in vitro.

2.3 Visualization of Photocontrollable Amyloidogenesis In Vitro

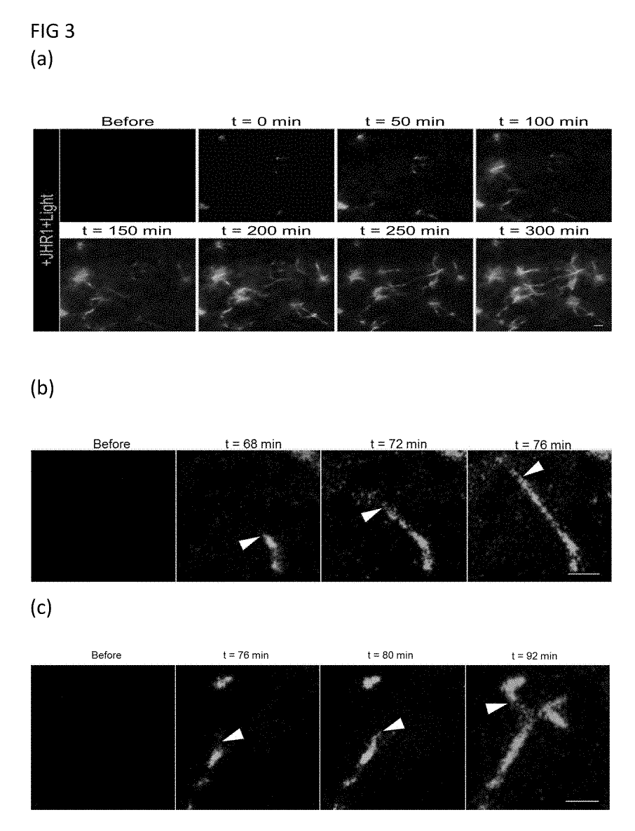

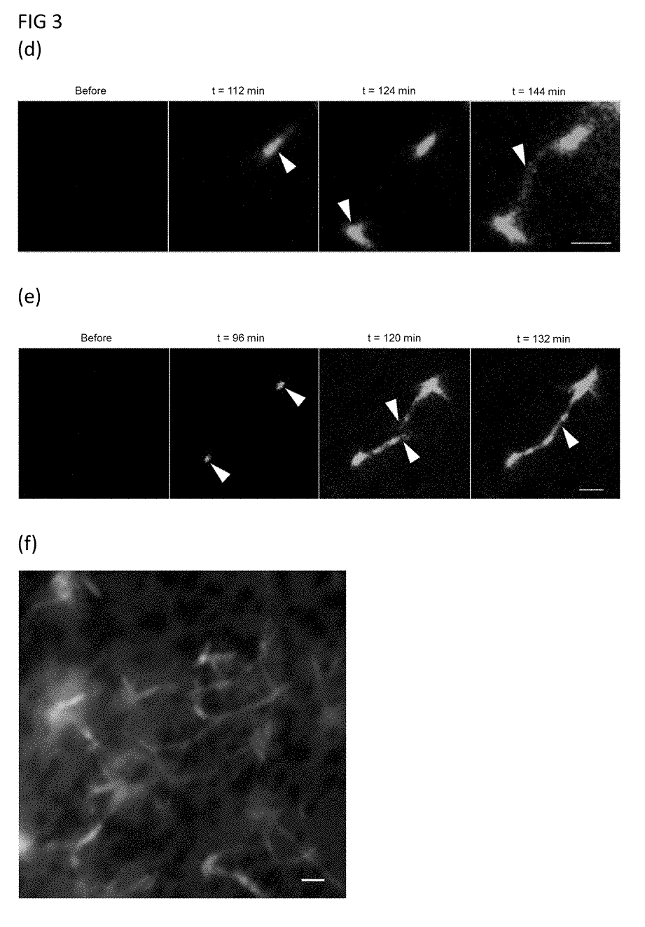

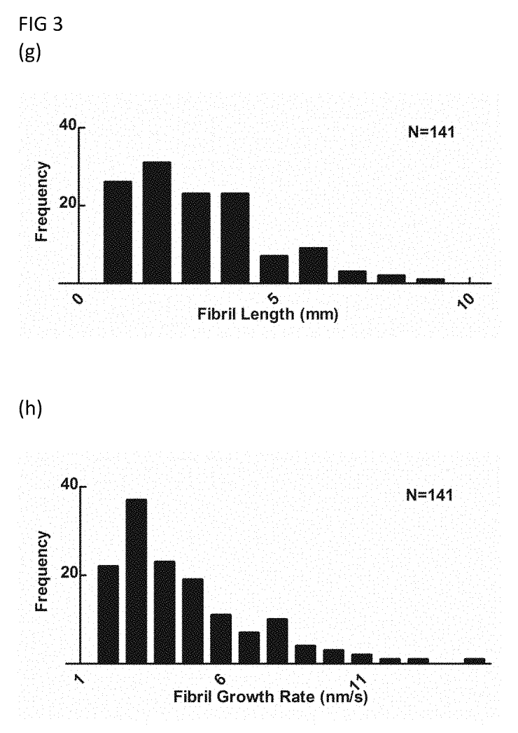

In this example, total internal reflection fluorescence (TIRF) microscopy was employed to directly observe the fibrillization kinetics of JHR1 upon photocleavage. Briefly, JHR1 was co-incubated with ThT and amyloid formation was observed at the excitation wavelength of 442 nm and emission wavelength of 485 nm. Results are depicted in FIG. 3.

it is generally believed that the kinetics of amyloid formation include the lag (nucleation), elongation (fibrillization), and final plateau phase; and as the photographs in FIG. 3, panel a depicted, JHR1 immediately gave rise to fluorescent “nuclei” after UV illumination followed by the formation of amyloid fibers and finally reached a plateau after 300 min. With higher magnification, the fibrillar growth patterns could be classified as either unidirectional growing end elongation (highlighted with arrowheads, FIG. 3, panel b), multidirectional branching of the budding termini (FIG. 3, panel c), linkage and fusion of the two growing ends (FIG. 3, panel d), or meeting of detour fibrillar ends (FIG. 3, panel e). Moreover, the merged images from 2 hr (green) and 5 hr (red) revealed that the newly established fibril network (in red) grew from the existing fibrils (yellow, merged) over 3 hr (FIG. 3, panel f). In the plateau phase, the mature fibril length (N=141) ranged from 1-9 μm (FIG. 3, panel g), and the average growth rate for the entire process was approximately 3.7 nm/s (FIG. 3, panel h). Unlike findings in previous reports, where the nucleation usually requires preformed fibrils as “nuclei” (Ogi H et al., Scientific reports 4, 6960 (2014)), the present JHR1 spontaneously forms “nuclei” allows rapidly “seeding” more monomers into amyloid after photo-initiation.

### Example 3 JHR2 Co-Localized with Cytosolic TDP-43 Aggregates

In this example, JHR2 of example 1 (which comprised a fluorophore-Alexa568 lined to the C-terminal cysteine side chain of TDP-43₃₀₇₋₃₂₂) (FIG. 1, panel a) was loaded into N2A cells so as to monitor the amyloidogenesis in a live cell.

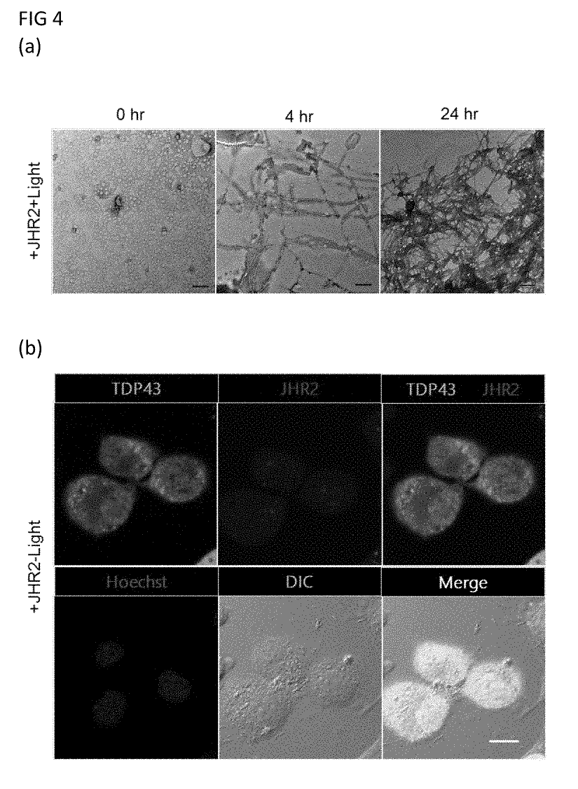

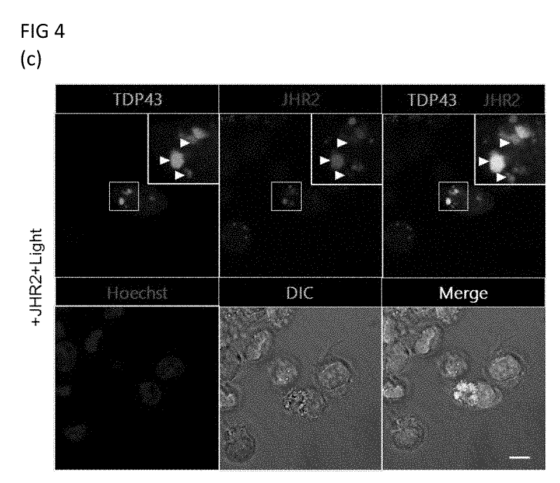

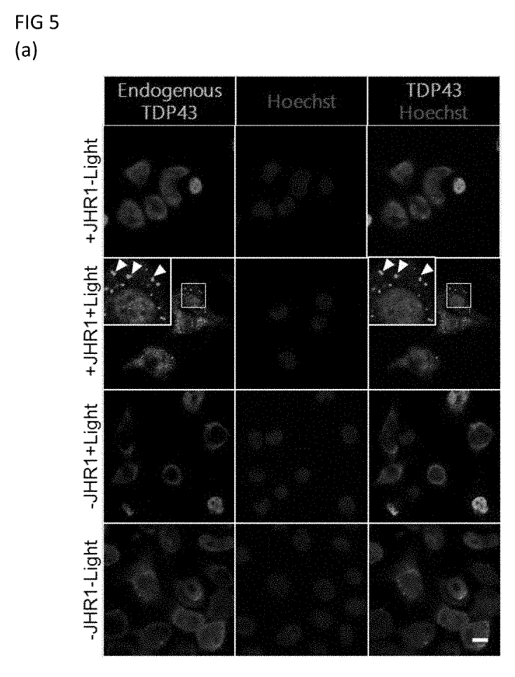

Upon illumination (wavelength: 335-379 nm, power density: 8.24 mW/cm², duration: 3 mins), massive fibrils were detected within 4 hr and an elaborate network of mature fibers was observed at later time points (FIG. 4, panel a). Sulfonated fluorophore (Alexa 568) was chosen to ensure the fluorescence emission spectrum, before and after induced aggregation, is identical (data not shown). N2A cells are mouse neuroblastoma cell line and commonly used as cellular model in the study of neurodegenerative diseases. To optimize the irradiation conditions in N2A cellular experiments, we had also found that no significant cell damage occurred in the non-focused light (wavelength: 335-379 nm, power density: ≦8.24 mW/cm², duration: ≦10 min) as confirmed by both DIC morphology observation and Annexin V staining experiments (data not shown). On the other hand, significant cell damage was observed in focused light (wavelength: 335-379 nm, power density: 31.5 mW/cm², duration: ≧1 min) with shrinkage morphology and strong Annexin V response (data not shown).

Results from the present example indicated that the fluorescent JHR2 may quickly penetrate cells (FIG. 4, panel b). Given that activated AMP-dependent protein kinase (AMPK) readily elicits TDP-43 mislocalization, an AMPK activator, 5-aminoimidazole-4-carboxamide ribonucleotide (AICAR), was employed to induce TDP-43 translocation to the cytosol, mimicking the early ALS pathology. After overnight incubation with AICAR and JHR2 in the dark, expressed EGFP-TDP-43 redistributed from mainly in the nucleus to the entire cell (FIG. 4, panel b). It should be noted that 24 hrs after a short irradiation with non-focused light (wavelength: 335-379 nm, power density: 8.24 mW/cm², duration: 3 mins), both JHR2 and EGFP-TDP-43 dramatically developed pronounced aggregates and/or inclusion bodies that were largely co-localized in the cytosol (FIG. 4, panel c), which implicated the photoactivated JHR2 to induce the soluble TDP-43 into the insoluble species.

### Example 4 Photocontrollable Probes “Seed” Endogenous TDP-43 and Cause Primary Cortical Neuron Degeneration Upon Irradiation

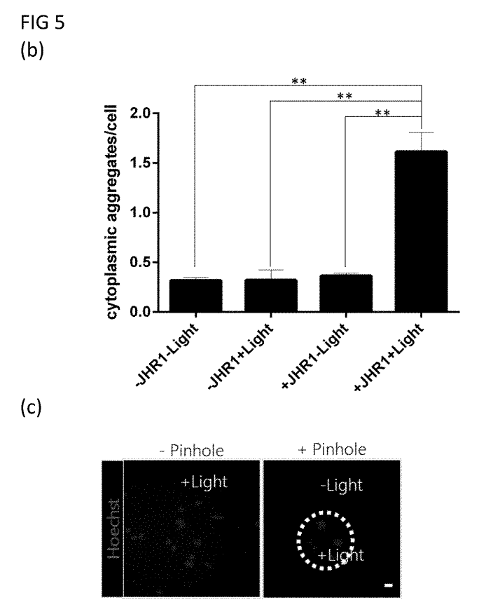

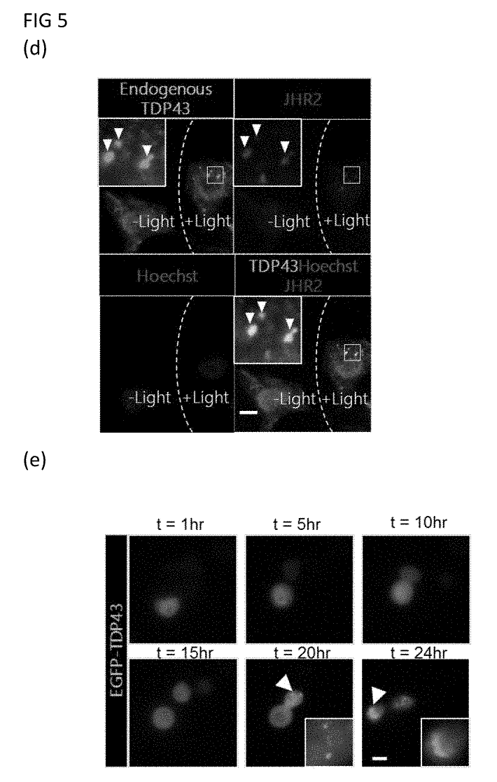

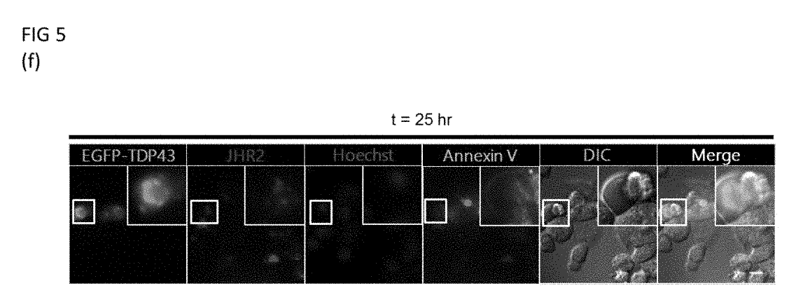

In this example, the spatial distribution of “endogenous TDP-43” within live cells is investigated. Accordingly, endogenous TDP-43 was immunostained with TDP-43-specific antibody after photoirradiation (FIG. 5, panel a). Endogenous TDP-43 aggregates (indicated by white arrows) were readily observed in the cytoplasm of N2A cells when both peptides and irradiation were applied (FIG. 5, panels a, b). Spinning disk confocal microscopy was used (see online methods) to precisely count the induced cytosolic TDP-43 aggregates. The average number of endogenous TDP-43 aggregates per cell was significantly higher with light and JHR1 (FIG. 5, panel b).

To demonstrate the intercellular spatial resolution of this platform, a specific pinhole in the optical path was applied to confine the UV illumination area (white circle in FIG. 5, panel c) and selectively activate the JHR2 within the light spot. The Hoechst fluorescence could only be observed within this region. Within the light spot, we selectively induced the JHR2 amyloid to “seed” endogenous TDP-43 cytosolic aggregation of different cells by spatially controlling the illuminated area (FIG. 5, panel d). To depict the entire dynamic interaction process in detail and real-time, cells expressing EGFP-TDP-43 were incubated with JHR2, and the time-course epi-fluorescence images were acquired. We observed that both EGFP-TDP-43 and JHR2 were mainly diffused within cells in the early incubation period (1-15 hr) (FIG. 5, panel e). Twenty hours after illumination, the EGFP-TDP-43 puncta became detectable and was more prominent at 24 hr (FIG. 5, panel e). By the end of the time course (25 hr), the N2A cells with the inclusion bodies of EGFP-TDP-43 in the cytoplasm were found to be on the verge of apoptosis, as revealed by the notable Annexin V staining (FIG. 5, panel f), which was consistent with pathological models of ALS.

To confirm that the aggregation of EGFP-TDP-43 exclusively correlated with the amyloidogenesis of JHR2 within the cytoplasm, N2A cells were microinjected with JHR2 and irradiated. The results recapitulated a similar time-dependent photoinduced EGFP-TDP-43 aggregation and the concomitant cell death yet with a shorter response time, suggesting that the octaarginine-directed peptide delivery efficiency was comparable to direct microinjection (data not shown). It was worth noting that the aggregation of EGFP-TDP43 (4 hr) always initiates after the clustering of JHR2 (2 hr) (data not shown).

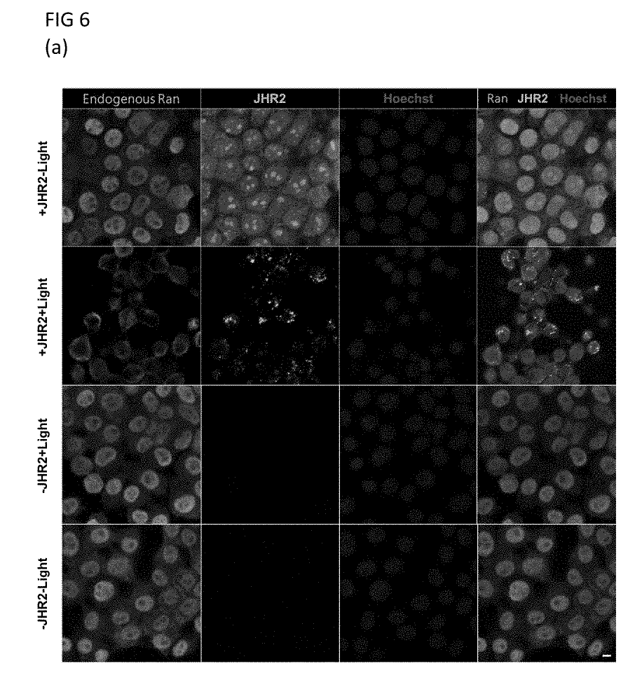

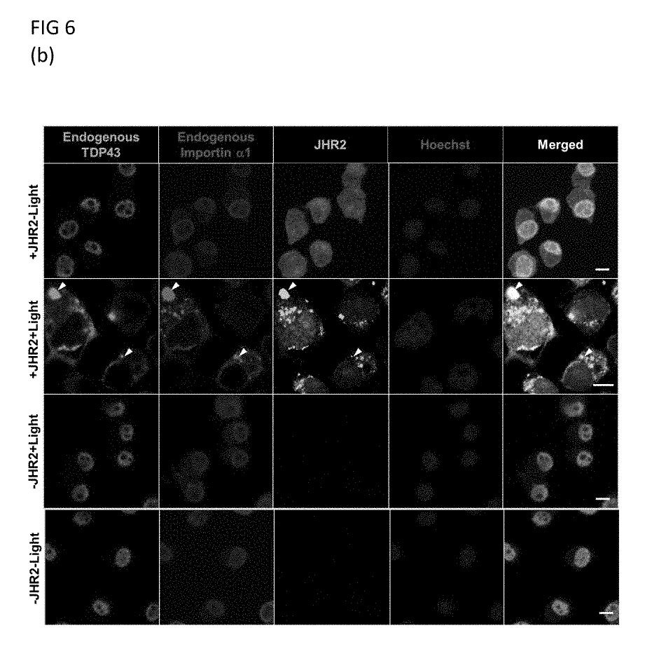

It has been reported that the accumulation of artificial proteins in the cytoplasm rather than in the nucleus could interfere with nucleocytoplasmic protein and RNA transport to induce cytotoxicity. Although it was suggested that multiple factors of the nuclear import and export machinery may be sequestered by the misfolded protein aggregates, the detailed hypothesized mechanism for this result remained unexplored. Since the present platform may selectively induce “photocontrollable seeding” in the cytosol to trigger endogenous TDP-43 aggregation, it would be advantageous to use the probes of the present disclosure to test the above hypothesis. Thus, the localization of Ran (RAS-related nuclear protein; a 25 kDa protein involved in transportation into and out of the cell nucleus) in the JHR2-treated or control cells in the absence and presence of “light” was investigated. The data indicated that Ran mainly resided in the nucleus in the untreated (−JHR2−Light), UV-irradiated only (−JHR2+Light), and JHR2-treated only (+JHR2−Light) cells (FIG. 6, panel a). However, upon illumination (+JHR2+Light), diffused JHR2 formed significant small puncta with more that 90% of Ran protein miss-localized into the cytoplasm (FIG. 6, panel a), suggesting the interference of nuclear shuttling by JHR2 aggregates. In addition to Ran, the impact of in vivo amyloidogenesis on the importin-α1 was investigated. Importin-α1 is the binding partner of Ran and may transport proteins containing the nuclear localization sequence (NLS). It was found that importin-α1 mainly remained in the nucleus in the untreated (−JHR2−Light), UV-irradiated only (−JHR2+Light), and JHR2-treated only (+JHR2−Light) cells (FIG. 6, panel b). On the contrary, TDP-43 and importin-α1 translocated from the nucleus to cytosol and formed aggregates in the presence of JHR2 under photoactivation (+JHR2+Light). Further, importin-α1 was found to be enriched around the nuclear periphery during the observation (FIG. 6, panel b). Taken together, the data indicated that JHR2 aggregates triggered the miss-localization of Ran, the enrichment of importin-α1 around the nuclear periphery, and the aggregation of importin-α1, which hint on the impaired nuclear-cytoplasmic transport by the cytoplasmic aggregates.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of the present disclosure.

What is claimed is:

1. A photocontrollable probe comprising in sequence:
a cell penetrating moiety, a photocleavable moiety, an amyloidogenic moiety, a fluorophore linking moiety and a fluorophore, wherein, the photocleavable moiety is labile upon exposure to a radiation having a wavelength between 250 to 1,000 nm.

2. The photocontrollable probe of claim 1, wherein the fluorophore linking moiety is a cysteine residue.

3. The photocontrollable probe of claim 1, wherein the fluorophore is a fluorescent dye selected from the group consisting of, aminocoumarin, allophycocyanin (APC), Alexa 488, Alexa 568, CY3, CY5, CY7, APC-CY7 conjugate, fluorescein, FluorX, hydroxycoumarin, lucifer yellow, methoxycoumarin, [2-(4-nitro-2,1,3-benzoxadiazol-7-yl)aminoethyl]trimethylammonium (NBD-TMA), phycoerythrin (PE), PE-CY5 conjugate, PE-CY7 conjugate, rhodamine, tetramethylrhodamine isothiocyanate (TRITC)-amines, and Texas Red amines.

4. The photocontrollable probe of claim 1, wherein the cell penetrating moiety comprises at least 2 charged amino acid residues selected from the group consisting of arginine, lysine, histidine, aspartate, glutamate, and a combination thereof.

5. The photocontrollable probe of claim 4, wherein the cell penetrating moiety is composed of 8 arginine residues.

6. The photocontrollable probe of claim 4, wherein the cell penetrating moiety is a cell penetrating peptide (CPP) selected from the group consisting of, Tat peptide, penetratin, pVEC, transportan, MPG, Rep-1, and R₆W₃.

7. The photocontrollable probe of claim 1, wherein the amyloidogenic moiety is an amyloidogenic peptide derived from amyloid-β (Aβ), amyloid islet protein, α-synuclein, mutant huntingtin, chromosome 9 open-reading frame 72 (C9orf72) dipeptide repeat protein (DRP), or TAR-DNA binding protein 43 (TDP-43).

8. The photocontrollable probe of claim 7, wherein the amyloidogenic peptide is derived from TDP-43 and has at least 90% sequence identity to SEQ ID NO: 2.

9. The photocontrollable probe of claim 7, wherein the amyloidogenic peptide is derived from C9orf72 DRP.

10. The photocontrollable probe of claim 9, wherein the amyloidogenic peptide derived from C9orf72 DRP is a poly(glycine-alanine) (poly-GA) fragment, a poly(glycine-proline) (poly-GP) fragment, or a poly(glycine-arginine) (poly-GR) fragment.

11. The photocontrollable probe of claim 7, wherein the amyloidogenic peptide is derived from mutant huntingtin.

12. The photocontrollable probe of claim 11, wherein the amyloidogenic peptide derived from mutant huntingtin is a polyglutamine (PolyQ) fragment

13. The photocontrollable probe of claim 7, wherein the amyloidogenic peptide is further conjugated with polyethylenimine (PEI).

14. The photocontrollable probe of claim 1, wherein the photocleavable moiety comprises a functional group selected from the group consisting of, arylcabonyl, nitroaryl, coumarin-4-yl-methyl, arylmethyl, a metal-containing group, arylsulfonyl, and a silicon based group.

15. The photocontrollable probe of claim 14, wherein the metal-containing group is [M(bpy)ₙ]²⁺, where M is Fe²⁺, Ru²⁺, or Co²⁺; n is 2 or 3; and bpy is 2,2′-bipyridine.

16. The photocontrollable probe of claim 14, wherein the silicon based group is tris(trimethylsilyl) group.

17. The photocontrollable probe of claim 14, wherein the photocleavable moiety has the structure of rose Bengal, riboflavin, or cobalamin.

18. The photocontrollable probe of claim 14, wherein the photocleavable moiety comprises the nitroaryl group, and is cleaved upon exposure to the radiation of 365 nm.

19. A method of determining the presence or absence of an amyloidogenic protein in a tissue or an organ of a subject, comprising:
(a) treating the tissue or the organ with the photocontrollable probe of claim 1;
(b) irradiating the photocontrollable probe-treated tissue or organ of the step (a) with a light having a wavelength between 250 to 1,000 nm; and
(c) subjecting the irradiated tissue or organ of the step (b) to an imaging analysis;
wherein, if amyloid fibrils were observed in the irradiated tissue or organ during the imaging analysis in the step (c), then the amyloidogenic protein is present in the tissue or the organ of the subject.

20. The method of claim 19, wherein in the step (b), the photocontrollable probe-treated tissue or organ is irradiated with the light having a wavelength of 365 nm.

21. The method of claim 19, wherein the amyloidogenic protein is any of Aβ, amyloid islet protein, α-synuclein, mutant huntingtin, C9orf72 DRP, or TDP-43.

22. The method of claim 19, wherein the amyloidogenic protein in the tissue or the organ is resulted from a neurodegenerative disease or diabetes mellitus.

23. The method of claim 19, wherein the organ is the brain, and the subject is a human.

24. The method of claim 23, wherein the neurodegenerative disease is scrapie, amyotrophic lateral sclerosis (ALS), Parkinson disease (PD), Alzheimer disease (AD), Lewy body disease, Huntington disease (HD), Machado-Joseph disease, dentatorubral pallidoluysian atrophy (DRPLA), spinal and bulbar muscular atrophy, spinocerebellar ataxia, or fragile X-associated tremor/ataxia syndrome.

25. The method of claim 22, wherein the diabetes mellitus is type II diabetes mellitus.


SEQUENCE LISTING (10 sequences)

SEQ ID NO: 1 - protein, 8 aa, Artificial Sequence
  cell penetration sequence
RRRRRRRR

SEQ ID NO: 2 - protein, 16 aa, Artificial Sequence
  amino acid residues 307 to 322 of TDP-43
MGGGMNFGAFSINPAM

SEQ ID NO: 3 - protein, 13 aa, Artificial Sequence
  Tat peptide
GRKKRRQRRRPPQ

SEQ ID NO: 4 - protein, 16 aa, Artificial Sequence
  penetratin
RQIKIWFQNRRMKWKK

SEQ ID NO: 5 - protein, 18 aa, Artificial Sequence
  pVEC
LLIILRRRIRKQAHAHSK

SEQ ID NO: 6 - protein, 27 aa, Artificial Sequence
  transportan
GWTLNSAGYLLGKINLKALAALAKKIL

SEQ ID NO: 7 - protein, 27 aa, Artificial Sequence
  MPG
GALFLGFLGAAGSTMGAWSQPKKKRKV

SEQ ID NO: 8 - protein, 21 aa, Artificial Sequence
  Rep-1
KETWWETWWTEWSQPKKKRKV

SEQ ID NO: 9 - protein, 18 aa, Artificial Sequence
  MAP
KLALKLALKALKAALKLA

SEQ ID NO: 10 - protein, 10 aa, Artificial Sequence
  R6W3
RRWWRRWWRR